(19) United States
(12) Patent Application Publication
(10) Pub. No.: US 2016/0296182 A1
(43) Pub. Date: Oct. 13, 2016

(54) FLAT PANEL X-RAY IMAGING DEVICE - TWIN DUAL CONTROL GUI

(71) Applicant: SCANFLEX HEALTHCARE AB, Stockholm (SE)

(72) Inventor: Volker NEUMANN, Hamburg (DE)

(21) Appl. No.: 15/037,725

(22) PCT Filed: Nov. 19, 2014
(86) PCT No.: PCT/SE2014/051381

§ 371 Date: May 19, 2016

Related U.S. Application Data
(60) Provisional application No. 61/906,043, filed on Nov. 19, 2013.

(51) Int. Cl. A61B 6/00; A61B 6/04

(52) U.S. Cl. CPC A61B 6/4014; A61B 6/487; A61B 6/4405; A61B 6/463; A61B 6/4476; A61B 6/0457; A61B 6/4441; A61B 6/467; A61B 2560/0295

(57) ABSTRACT

A mobile digital fluoroscopy system, having a mobile unit (1) having a first and a second X-ray system each having a transmitter (21, 23) and a receiver (22, 24), said respective first and second X-ray systems to enable X-ray imaging in mutually intersecting planes comprising a first control unit (510) configured with a control interface (520) and a memory (530), wherein said control interface comprises multiple disjunctive control interface windows of the control interface adapted to display data based on functional status data, wherein said first control unit is further configured to receive functional status data as status control signals from receivers (22, 24), process function status data to a visual representation of said function status data as display data, present said visual representation on said control interface 520 based on said display data, wherein presenting comprises displaying subsets of display data in said multiple disjunctive control interface windows (610-690), wherein said multiple disjunctive control interface windows comprises, a first disjunctive control interface window adapted to display a first set of display data obtained as function status data in the form of image data captured by said first X-ray system, a second control interface window adapted to display a second set display data obtained as function status data in the form of image data captured by said second X-ray system and a third control interface window adapted to display a third set of display data obtained as functional data received from said memory in the form of a scrollable thumbnail list, wherein the functional status data comprises a thumbnail representation of captured X-ray image data and associated information.

### FIELD OF THE INVENTION

The present invention relates in general to a preferably mobile digital fluoroscopy system for medical applications operating with an X-ray device mounted to generate X-ray images.

More specifically, the present invention relates to a fluoroscopy system having multiple X-ray devices each provided with a flat digital X-ray detector, and oriented on different axes to provide different views of the area of interest in the patient with the ability to provide for patient data management, multiplanar reconstruction, segmentation, image measurements, image export, and image print, view and storage to transitory media or memory.

### BACKGROUND

In orthopedic surgery environment, there is a need for allowing full access to the operating area with total control at each step. Therefore, X-ray imaging using C-stands or G-stands comprising imaging systems is commonly used, wherein a C-stand only has one X-ray imaging device while a so-called G-stand comprises two such imaging devices, with their axes oriented at an angle to each other.

A symmetrical G-stand is generally preferable to a C-stand, since it comprises two perpendicularly mounted X-ray imaging devices, and is thereby able to provide both frontal and lateral X-ray imaging with fixed settings. The ability to simultaneously see the surgical area in both a frontal and lateral view reduces the need to move and adjust the equipment during surgery, thus reducing both surgery time and radiation dose. When the need to move the equipment is reduced, better sterility is also achieved.

The ability in a G-stand to double the surgeon's view also results in accurate positioning of implants, creating a safer and more reliable method of surgery. The X-ray devices are fixed in perpendicular relation to each other in the G-stand, but the entire G-stand can be tilted somewhat for better access and views. Or in some G-stand systems, the G-stand is somewhat rotatable about a horizontal axis perpendicular to the axes of both of the X-ray devices.

A problem, in particular when performing surgery and using the X-ray system, is that the area of interest and related information must be relevant for the current procedure and aggregate data in an optimal manner.

### SUMMARY OF THE INVENTION

The general object of the invention is to provide improvements in a digital fluoroscopy system for medical applications operating with first and second X-ray imaging devices mounted on a G-stand to generate X-ray images along two mutually intersecting axes.

The general inventive concept of the invention is for 2D and 3D visualization of X-ray images during surgery, e.g. of the spine and other structures.

The system may be a mobile or a stationary Fluoroscopy X-ray imaging system used by physicians during surgery and being configured to generate X-rays in two more intersecting planes. Such a system may be configured on carrier comprising a section of ring or other configuration with the X-ray sources and image acquisition mounted thereon, and a first control unit containing power electronics, display monitors, a computer for controlling the system, a touch-screen with a GUI for the user to control the use of the system.

One aspect of the invention provides for patient data management, multiplanar reconstruction, segmentation, image measurements, image export, and image print, view and storage to transitory media or memory.

Yet another aspect of the invention provides the capability to overlay annotations on 2D medical image displays as well as other special image measurements and evaluations

A problem with conventional systems is to adapt the presentation of X-ray images and associated data as the user requirements changes, e.g. during different phases of surgery relying on X-ray imaging.

An embodiment of the invention comprises a mobile digital fluoroscopy system, having a mobile unit (1) having a first and a second X-ray system each having a transmitter (21, 23) and a receiver (22, 24), said respective first and second X-ray systems being configured to enable X-ray imaging in mutually intersecting planes, comprising:

a first control unit (510) configured with a control interface (520) and a memory (530) herein also called data storage, wherein said control interface comprises a plurality of disjunctive control interface windows of the control interface adapted to display controls configured to control functions of the fluoroscopy system and to display data based on functional status data of said first and second X-ray systems. The displayed controls may be symbols of varying complexity adapted to realize control means adjustable through input signals via a touch screen comprised in the control interface.

In an embodiment of the invention, a mobile digital fluoroscopy system, having a mobile unit (1) having a first and a second X-ray system each having a transmitter and a receiver, said respective first and second X-ray systems being mounted on a G-arm to enable X-ray imaging in mutually intersecting planes;

comprising

a first control unit configured with a control interface and a memory, wherein said control interface comprises multiple disjunctive control interface windows of the control interface adapted to display data based on display data, wherein said first control unit is further configured to:

receive functional status data as status control signals from a selection of said memory, a servo motor unit, a third control unit and a fourth control unit;

process function status data to a visual representation of said function status data as display data

present said visual representation based on said display data.

In an embodiment of the invention, wherein said multiple, multiple herein also called a plurality of, disjunctive control interface windows, windows herein also called data presentation fields, comprises:

a first control interface window adapted to display a first set of display data obtained as function status data in the form of image data captured by said first X-ray system;

In an embodiment of the invention, wherein said control interface further comprises a seventh control interface window adapted to display a seventh set display data obtained as function status data in the form of register data associated with said first X-ray system obtained from memory

In an embodiment of the invention, wherein said register data associated with said first X-ray system comprises register cards further comprising register card specific functions, wherein said register card specific function is a selection of:

a view function configured to provide the user access to image viewing specific functions such as orientation and zoom. Print, Save Image and Save Cine

an image settings function configured to provide the user access to image setting specific functions such as control of auto windowing, edge enhancement, brightness and contrast;

an annotation function configured to provide the user access to annotation specific functions such as placing markers and measure length and angle in the image;

an image sequence function configured to provide the user access to image sequence replay specific functions such as playback functions for cinematic loop playback.

In an embodiment of the invention, wherein said multiple disjunctive control interface windows comprises:

a second control interface window adapted to display a second set display data obtained as function status data in the form of image data captured by said second X-ray system;

In an embodiment of the invention, wherein said control interface further comprises an eighth control interface window adapted to display a eighth set display data obtained as function status data in the form of register data associated with said second X-ray system obtained from memory

In an embodiment of the invention, wherein said register data associated with said second X-ray system comprises register cards further comprising register card specific functions, wherein said register card specific function is a selection of:

a view function configured to provide the user access to image viewing specific functions such as orientation and zoom. Print, Save Image and Save Cine

an image settings function configured to provide the user access to image setting specific functions such as control of auto windowing, edge enhancement, brightness and contrast;

an annotation function configured to provide the user access to annotation specific functions such as placing markers and measure length and angle in the image;

an image sequence function configured to provide the user access to image sequence replay specific functions such as playback functions for cinematic loop playback.

In an embodiment of the invention, wherein said multiple disjunctive control interface windows comprises:

a fourth control interface window adapted to display a fourth set of display data obtained as a first subset of said functional data received from said first X-ray system

In an embodiment of the invention, wherein said multiple disjunctive control interface windows comprises:

a fifth control interface window adapted to display a fifth set of display data obtained as a second subset said functional data received from said second X-ray system

In an embodiment of the invention, wherein said multiple disjunctive control interface windows comprises:

a sixth control interface window adapted to display a sixth set of display data obtained as a third subset of functional data received from said first X-ray system and from said second X-ray system and not part of said first or second subset;

In an embodiment of the invention, wherein said multiple disjunctive control interface windows comprises:

a ninth control interface window adapted to display a ninth set of display data obtained as functional data received from a fourth control unit indicative of the position of transmitter/receiver pairs on the mobile unit and/or the relative position of the object being subjected or the X-ray-beam;

In an embodiment of the invention, wherein said multiple disjunctive control interface windows comprises:

a ninth control interface window adapted to display a ninth set of display data obtained as functional data received from said memory in the form of a scrollable thumbnail list, wherein the functional status data comprises a thumbnail representation of captured X-ray image data and associated information

In an embodiment of the invention, a method in a mobile digital fluoroscopy system, having a mobile unit (1) having a first and a second X-ray system each having a transmitter and a receiver, said respective first and second X-ray systems being mounted on a G-arm to enable X-ray imaging in mutually intersecting planes;

Comprising the steps:

receive functional status data as status control signals from a selection of said memory, a servo motor unit, a third control unit and a fourth control unit;

process function status data to a visual representation of said function status data as display data

present said visual representation based on said display data.

In an embodiment of the invention, further comprising:

display a first set of display data obtained as function status data in the form of image data captured by said first X-ray system in a first control interface window;

In an embodiment of the invention, further comprising:

display a seventh set display data obtained as function status data in the form of register data associated with said first X-ray system obtained from memory in a seventh control interface window

In an embodiment of the invention, wherein said register data associated with said first X-ray system comprises register cards further comprising register card specific functions, wherein said register card specific function is a selection of:

a view function configured to provide the user access to image viewing specific functions such as orientation and zoom. Print, Save Image and Save Cine

an image settings function configured to provide the user access to image setting specific functions such as control of auto windowing, edge enhancement, brightness and contrast;

an annotation function configured to provide the user access to annotation specific functions such as placing markers and measure length and angle in the image;

an image sequence function configured to provide the user access to image sequence replay specific functions such as playback functions for cinematic loop playback.

In an embodiment of the invention, wherein said multiple disjunctive control interface windows comprises:

a second control interface window adapted to display a second set display data obtained as function status data in the form of image data captured by said second X-ray system;

In an embodiment of the invention, wherein said control interface further comprises an eighth control interface window adapted to display a eighth set display data obtained as function status data in the form of register data associated with said second X-ray system obtained from memory

In an embodiment of the invention, wherein said register data associated with said second X-ray system comprises register cards further comprising register card specific functions, wherein said register card specific function is a selection of:

a view function configured to provide the user access to image viewing specific functions such as orientation and zoom. Print, Save Image and Save Cine

an image settings function configured to provide the user access to image setting specific functions such as control of auto windowing, edge enhancement, brightness and contrast;

an annotation function configured to provide the user access to annotation specific functions such as placing markers and measure length and angle in the image;

an image sequence function configured to provide the user access to image sequence replay specific functions such as playback functions for cinematic loop playback.

In an embodiment of the invention, wherein said multiple disjunctive control interface windows comprises:

a fourth control interface window adapted to display a fourth set of display data obtained as a first subset of said functional data received from said first X-ray system

In an embodiment of the invention, wherein said multiple disjunctive control interface windows comprises:

a fifth control interface window adapted to display a fifth set of display data obtained as a second subset said functional data received from said second X-ray system

In an embodiment of the invention, wherein said multiple disjunctive control interface windows comprises:

a sixth control interface window adapted to display a sixth set of display data obtained as a third subset of functional data received from said first X-ray system and from said second X-ray system and not part of said first or second subset;

In an embodiment of the invention, wherein said multiple disjunctive control interface windows comprises:

a ninth control interface window adapted to display a ninth set of display data obtained as functional data received from a fourth control unit indicative of the position of transmitter/receiver pairs on the mobile unit and/or the relative position of the object being subjected or the X-ray-beam;

In an embodiment of the invention, wherein said multiple disjunctive control interface windows comprises:

a ninth control interface window adapted to display a ninth set of display data obtained as functional data received from said memory in the form of a scrollable thumbnail list, wherein the functional status data comprises a thumbnail representation of captured X-ray image data and associated information.

In an embodiment of the invention, a computer program product comprising computer readable code configured to, when executed in a processor, perform any or all of the method steps of claim 13-24.

In an embodiment of the invention, a non-transitory computer readable memory on which is stored computer readable code configured to, when executed in a processor, perform any or all of the method steps of claim 13-24.

In an embodiment of the invention, a mobile digital fluoroscopy system, having
a mobile unit (1) having a first and a second X-ray system each having a transmitter and a receiver, said respective first and second X-ray systems being mounted on a G-arm to enable X-ray imaging in mutually intersecting planes;

comprising:
a dose measuring device mounted on each respective X-ray transmitter and configured to measure emitted dose of X-ray radiation from said respective X-ray transmitter;
a dose calculating device configured to calculate a dose of X-ray radiation received by an object from said respective X-ray transmitter and according to one or more selected dose indication methods;
a graphical user interface having indicia presenting said received X-ray radiation dose.

In an embodiment of the invention, a computer program product comprising computer readable code configured to, when executed in a processor, perform any or all of the method steps described herein.

In an embodiment of the invention, a non-transitory computer readable memory on which is stored computer readable code configured to, when executed in a processor, perform any or all of the method steps described herein.

In an embodiment of the invention, a mobile digital fluoroscopy system, having
a mobile unit (1) having a first and a second X-ray system each having a transmitter and a receiver, said respective first and second X-ray systems being mounted on a G-arm to enable X-ray imaging in mutually intersecting planes;

comprising:
a heat management system configured to monitor heat generated by said respective X-ray transmitter and to control operational mode of said X-ray transmitters based on detected current heat condition and on predetermined rules;
a graphical user interface having indicia presenting a current heat status and possibly current operational mode of said X-ray detectors.

In an embodiment of the invention, a computer program product comprising computer readable code configured to, when executed in a processor, perform any or all of the method steps described herein.

In an embodiment of the invention, a non-transitory computer readable memory on which is stored computer readable code configured to, when executed in a processor, perform any or all of the method steps described herein.

The advantage of the invention is that an improved overview and control of an X-ray imaging system is obtained thereby improving quality of surgery, reducing risk in surgery and reducing time a patient will have to be exposed to X-ray radiation.

The invention solves this by aggregating data and function in a manner that reduces system re-configuration time when user requirements changes, e.g. by allowing a surgeon to switch between live X-ray imaging and reference images obtained at a previous time instant.

### BRIEF DESCRIPTION OF THE DRAWINGS

The present invention will be further explained below with reference to the accompanying drawings, in which:

FIG. 1 shows a schematic overview of an exemplifying embodiment of the invention in a digital fluoroscopy system configured on a G-arm on a mobile G-stand coupled to a mobile first control unit;

FIG. 2 shows a schematic view of an exemplifying embodiment of a mobile first control unit;

FIG. 3 shows a schematic first view of an exemplifying embodiment of a control interface as a graphical user interface GUI implemented in the mobile first control unit;

FIG. 4 shows a schematic second view of an exemplifying embodiment of a control interface as a graphical user interface GUI implemented in the mobile first control unit.

FIG. 5 shows a schematic view of a first control unit, a servo motor unit, a third control unit and a fourth control unit.

FIG. 6 shows a schematic view of a control interface comprising multiple disjunctive control interface windows.

FIG. 7 shows an exemplary embodiment of a control interface comprising multiple disjunctive control interface windows

### DETAILED DESCRIPTION OF THE INVENTION

Introduction

When operating an X-ray imaging system, in particular during continuous use as when supporting surgery, there are a number of aspects that needs to be controlled:

presenting a captured X-ray image

adapting the presented image

selecting the image or images presented.

controlling X-ray beam generation

controlling position of the X-ray system and patient

managing patient data

controlling narrowing or collimation of the X-ray beam

By improving monitoring and control of the above mentioned aspects an improved efficiency and reduced procedure time can be achieved when operating the X-ray system, e.g. when performing surgery with the support of the X-ray system.

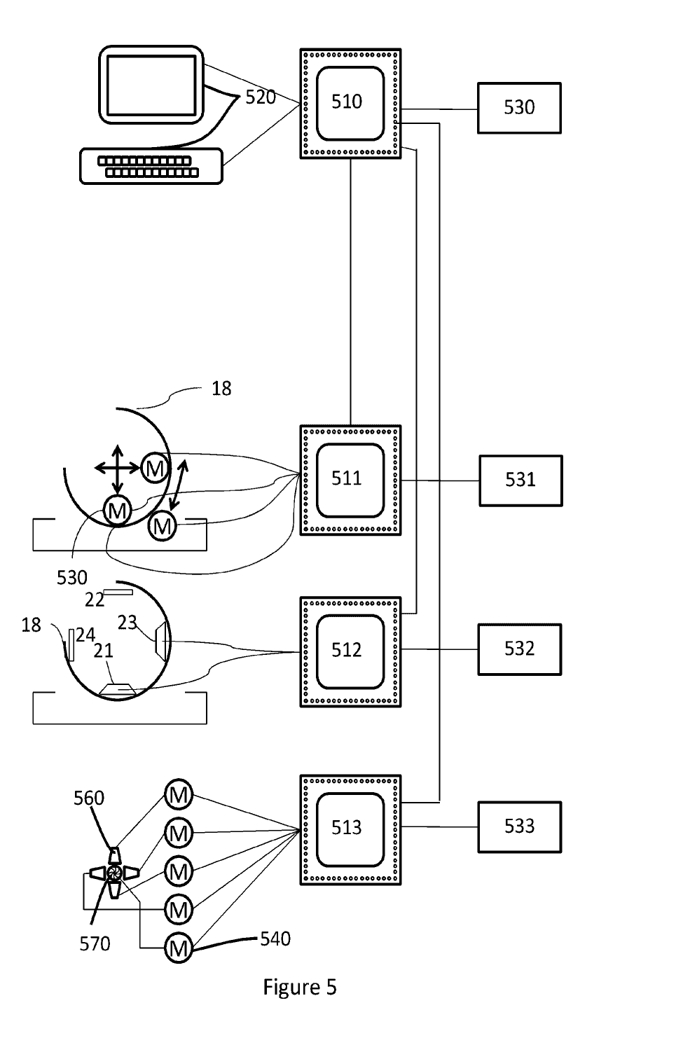

An embodiment of a digital fluoroscopy system is shown in FIG. 5, wherein the system comprises a first control unit 510, a fourth control unit 511, a third control unit 512, and a servo motor unit 513. X-ray images are obtained by a first control unit 510 by capturing an X-ray beam transmitted by a transmitter 21, (an X-ray tube or x-tube) for emitting X-rays, by a receiver 22, (e.g. image intensifier or semiconductor sensors) as captured image data, adapting the captured image data by image processing the captured image based on image settings to a visual representation and presenting the visual representation in a control interface window in a combined user indication input and display device 520, .e.g. a touchscreen. User indications are received by said first control unit as user input data, further discussed in relation to FIG. 5.

The X-ray beam transmitted is controlled by a third control unit 512 comprised in the transmitter. The third control unit is configured to determine the X-ray energy emitted by the transmitter based on predefined data parameters retrieved from a memory 532 communicatively coupled to said third control unit and user input data received from said first control unit 510. The third control unit is further configured to send third control data indicative of X-ray beam transmission to said first control unit 510. The third control unit 512 further determines the X-ray beam dose administered to an object, .e.g. a patient. In one embodiment, the third control unit is configured to determine the X-ray energy emitted by the transmitters based on predefined data parameters retrieved from a memory 532 communicatively coupled to said third control unit and functional status data as user input data received from said first control unit 510, to send functional status data indicative of X-ray beam transmission to said first control unit and to determine the X-ray beam dose administered to an object, .e.g. a patient.

The area of interest or area radiated by the X-ray beam may be controlled by narrowing the X-ray beam by the use of collimator plates 560 disposed between a beam transmitter and a beam receiver. Optionally the X-ray beam might be narrowed by the use of a dilatable collimator iris 570 disposed between a beam transmitter and a beam receiver. The control of the area of interest is achieved by the use of a servo motor unit 513 configured or adapted to receive control data as control signals from said first control unit, wherein the control data is based on processed user input data, to control servo motors 540 to a predetermined position based on said control data by sending servo motor signals, thereby narrowing the area of interest of the patient exposed to the X-ray beam. Servo motor status data indicative of the status of a servo motor is obtained by the servo motor unit by receiving servo motor signals and to send servo motor status data as status control signals to said control unit. In one embodiment the servo motor unit is configured to receive functional status data as control signals from said first control unit, wherein the control data is based on processed user input data, to control servo motors to a predetermined position based on said functional status data by sending servo motor signals, thereby narrowing the area of interest of the patient exposed to the X-ray beam and to obtain servo motor functional status data indicative of the status of a servo motor by receiving servo motor signals and to send servo motor functional status data as status control signals to said first control unit. The servo motor unit and the servo motors are communicatively coupled to each other, for instance by means of a cable or through wireless signal transmission. The servo motor unit and the control unit 2a are communicatively coupled to each other, for instance by means of a cable or through wireless signal transmission.

The position of transmitter/receiver pairs on the mobile unit and the relative position of the object being subjected or the X-ray-beam, e.g. a patient, may be controlled by sending control signals from said first control unit 510 to a fourth control unit 511. The position of transmitter/receiver pairs on the mobile unit, e.g. by rotating the arm 18 and the position of the object being subjected or the X-ray-beam is achieved by the use of said fourth control unit 511 configured or adapted to receive control data as control signals from said first control unit, wherein the control data is based on processed user input data, to control servo motors to a predetermined position based on said control data by sending servo motor signals, thereby positioning the transmitter/receiver pairs on the mobile unit or the object being subjected or the X-ray-beam. Servo motor status data indicative of the status of a servo motor is obtained by the fourth control unit 511 by receiving servo motor signals and to send servo motor status data as status control signals to said first control unit. In one embodiment, the fourth control unit 511 is configured to receive functional status data as control signals from said first control unit, wherein the functional status data is based on processed user input data, to control servo motors to a predetermined position based on said functional status data by sending servo motor signals, thereby positioning the transmitter/receiver pairs on the mobile unit or the object being subjected or the X-ray-beam. Servo motor functional status data indicative of the status, e.g. position of a servo motor, of a servo motor is obtained by the fourth control unit 511 by receiving servo motor signals and to send functional status data as status control signals to said first control unit. The servo motor unit and the servo motors are communicatively coupled to each other, for instance by means of a cable or through wireless signal transmission. The fourth control unit 511 and the first control unit 510/console 2a are communicatively coupled to each other, for instance by means of a cable or through wireless signal transmission.

An object, typically the body of a patient undergoing surgery, is placed inside the mobile unit 1 so that beam axis P1 and beam axis P2 of the two X-ray systems cross within the object. The first X-ray device 19 includes a first transmitter 21 (an X-ray tube or x-tube) for emitting X-rays and a first receiver 22 (e.g. image intensifier or semiconductor sensors) for receiving X-rays emitted by the first transmitter 21 and having passed through the object. The first transmitter 21 may be located down below on the arm 18 and the first receiver 22 at the top of the arm 18.

System Overview

The present invention concerns an X-ray apparatus configured as a system of components illustrated in the Figures of the drawings, adapted for use in connection with surgical orthopedic operations.

Embodiments of the invention comprise a mobile G-arm fluoroscopy system provided with flat digital X-ray detectors.

According to an embodiment, there is provided a mobile digital fluoroscopy system, comprising a mobile unit 1, also called a mobile X-ray system carrier unit 1, having a stand having a G-arm 18 suspended on a chassis frame 7; a first X-ray device 19 mounted on the G-arm 18 to transmit an X-ray beam along a first plane P1, the first X-ray device 19 having a first receiver 22 mounted on the G-arm 18 and a first transmitter 21 mounted on the G-arm 18 opposite said first receiver 22; a second X-ray device 20 mounted on the G-arm 18 to transmit an X-ray beam along a second plane P2 intersecting the first axis P1 of the first X-ray device, the second X-ray device 20 having a second receiver 24 mounted on the G-arm 18 and a second transmitter 23 mounted on the arm 18 opposite said second receiver 24, wherein said first and second receivers 22 and 24 are flat digital X-ray detectors mounted at respective ends of the G-arm.

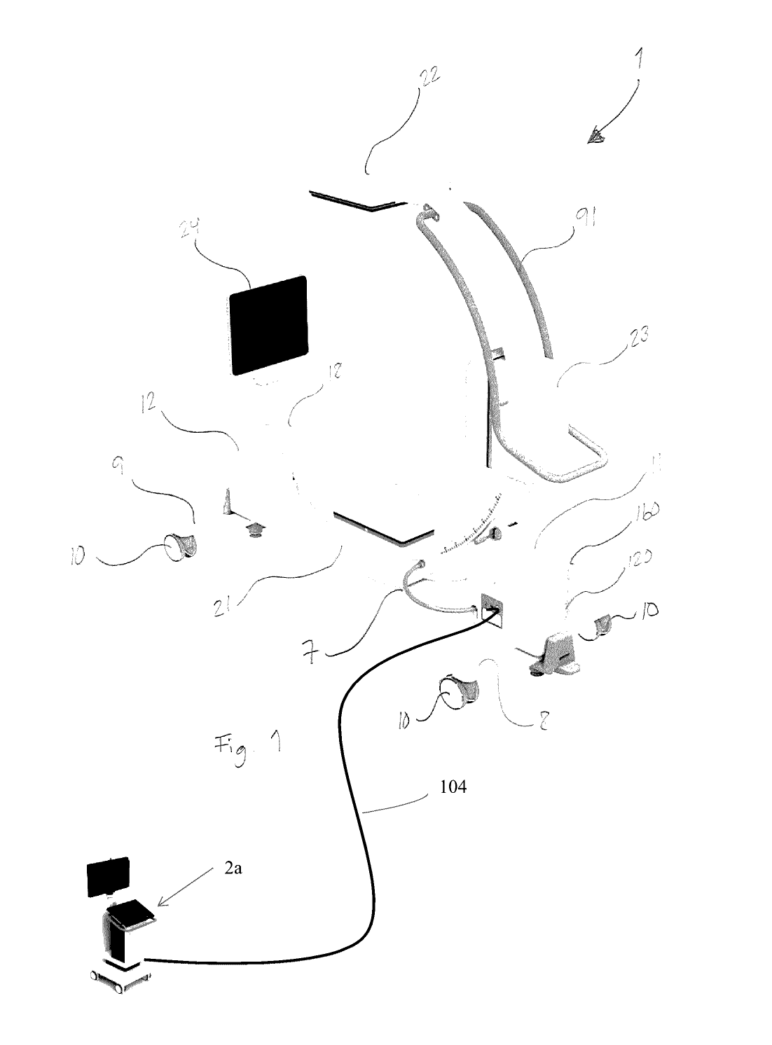

The apparatus shown in FIG. 1 comprises a mobile unit 1, i.e. a mobile X-ray system carrier unit 1 provided with two X-ray systems 19, 20 mounted to operate and transmit X-ray beams along mutually intersecting axes P1, P2. The arm 18 of the embodiment illustrated in FIG. 1 is referred to as a G-arm.

An object, typically the body of a patient undergoing surgery, is placed inside the mobile unit 1 so that beam axis P1 and beam axis P2 of the two X-ray systems cross within the object. The first X-ray device 19 includes a first transmitter 21 (an X-ray tube or x-tube) for emitting X-rays and a first receiver 22 (e.g. image intensifier or semiconductor sensors) for receiving X-rays emitted by the first transmitter 21 and having passed through the object. The first transmitter 21 may be located down below on the arm 18 and the first receiver 22 at the top of the arm 18. The second X-ray device 20 includes a second transmitter 23 (an X-ray tube or x-tube) for emitting X-rays and a second receiver 24 (e.g. image intensifier or semiconductor sensors) for receiving X-rays emitted by the second transmitter 23 and having passed through said object. The receivers 22, 24 may each comprise image intensifying means and an image capturing device, typically a CCD camera, for converting X-rays into a visible image.

FIG. 1 shows a G-arm to be placed around the patient together with a separate first control unit or console 2a which can be operated by the surgeon prior to the operation or during the operation by an assistant who does not have sterility restraints. High definition monitors 4a face the surgeon displaying the X-ray images in two different orthogonal planes either in real time or in so called “cine” replay to review exactly how and precisely where a prosthetic joint component has been placed without the necessity of exposing the patient and surgeon to ore X-ray radiation.

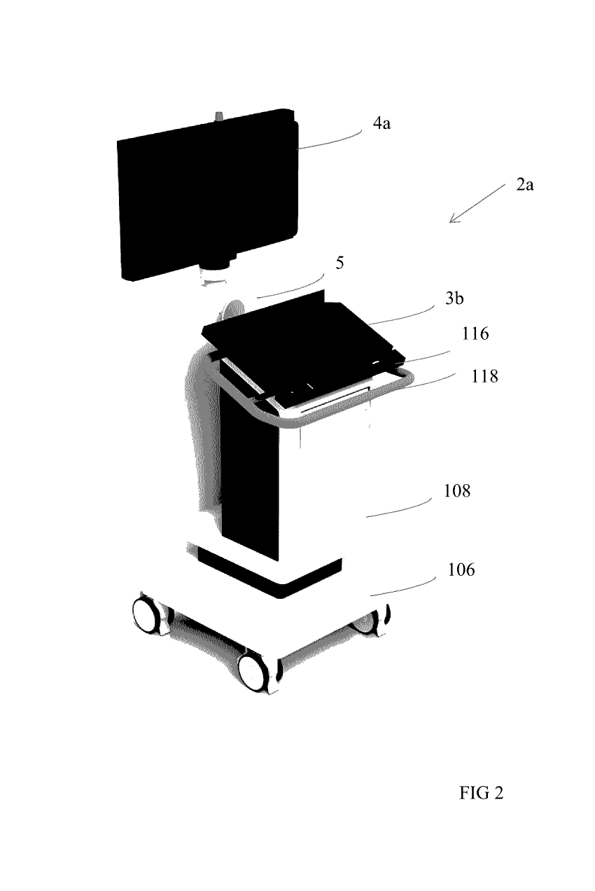

FIG. 2 shows an embodiment of a mobile first control unit 2a, also called console 2a, is provided with a base module 106 on wheels, a pulpit stand module 108 having a larger main part and a back part with a slot 5 in between. An operator control interface in the form of a touch screen 3b devised for presentation of one or more graphical user interfaces and a physical button panel 116 are mounted on the main part of the pulpit stand module to form a lectern like control panel, in this example also comprising a handle 118 configured for gripping when moving around the console and for resting to support ergonomic operation of the control interface. The back part of the pulpit stand module is configured for mounting display monitors or screens for presenting X-ray images.

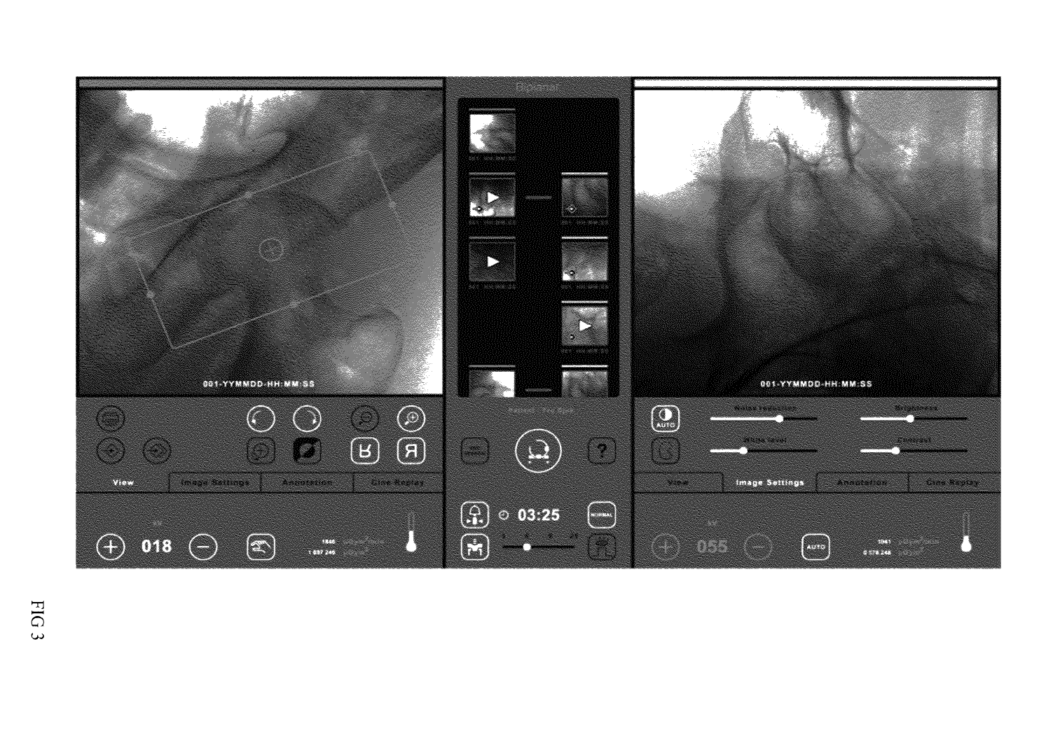

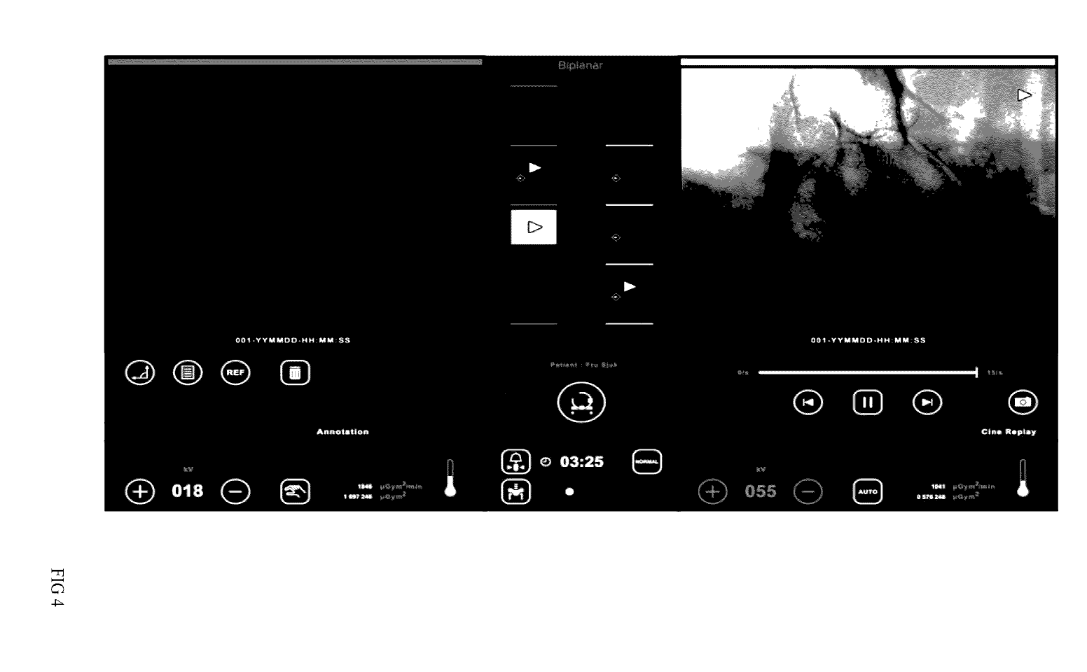

The HD display monitors 4a can be turned to face the user or operator of the console or can be turned to face a different direction. During an operation, the high definition monitors will typically be turned around to present the fluoroscopic images to the surgeon. The cables , seen in FIG. 1 104, connecting the G-stand to the console can be wound up and stored in the slot 5 when the console and the G-stand are close to each other. The console shown in FIG. 2 has a control interface, e.g. a touch screen graphic user interface (GUI) 3b, comprising in this case two fields which can be configured in various ways as shown in FIGS. 3-4. FIG. 3 for example shows horizontal and vertical X-ray views of a prosthesis mounted in a patient's hip, each view being surrounded by touch screen button or slide controls as well as numerical or analogue read-outs. The GUI may be presented with a configuration in which the left half of the touchscreen has a keyboard for inputting and recording information to identify patient or operation information for example and “cine” recordings.

Such a system may in addition to comprising high resolution monitors for presenting images to a surgeon for example also comprise components such as a foot switch (not shown) to enable the surgeon with sterile hands to switch between images taken in the respective planes. The first control unit preferably further comprises at least one touch screen display for displaying image data, a control panel, and a data processor comprising image processing means adapted to receive images transmitted from said image capturing devices comprised in said receivers 22, 24. The mobile unit is and the first control unit 2a are communicatively coupled to each other, for instance by means of a cable or through wireless signal transmission.

The first control unit is further configured to receive user indications via said touch screen as user input data in the form of user input data signals, to process user input data to control data indicative of a desired adjustments of functions in system, to send said control data as control signals to such functions, to receive functional status data as status control signals from a respective functions, to process function status data to a visual representation of said function status data and to send said visual representation to said touch screen as a display signal, wherein said touch screen is configured to display said visual representation to a user.

The first control unit further comprises a processor/processing unit 510 provided with specifically designed programming or program code portions configured to control the processing unit to perform the steps and functions of embodiments of the inventive method described herein. The first control unit further comprises at least one memory 530 configured to store data values or parameters received from a processor 510 or to retrieve and send data values or parameters to a processor 510. The first control unit further comprises a communications interface configured to send or receive data values or parameters to/from a processor 510 to/from external units, such as a servo motor unit 513, a third control unit 511 and a fourth control unit 512, via the communications interface.

In one or more embodiments the processor/processing unit 510 may be a processor such as a general or specific purpose processor/processing unit for example a microprocessor, microcontroller or other control logic that comprises sections of code or code portions, stored on a computer readable storage medium, such as a memory 530, that are fixed to perform certain tasks but also other alterable sections of code, stored on a computer readable storage medium, that can be altered during use. Such alterable sections of code can comprise parameters that are to be used as input for the various tasks, such as receiving user indications.

In one or more embodiments the first control unit further comprises a display configured to receive a display signal from a processor 510 and to display the received signal as a displayed image, e.g. to a user control.

In one or more embodiments the X-ray system further comprises an input device 520 , e.g. integrated in the touch screen, configured to receive input or indications from a user as user input data.

In one or more embodiments, wherein communications interface may include at least one of a Local Area Network (LAN), Metropolitan Area Network (MAN), Global System for Mobile Network (GSM), Enhanced Data GSM Environment (EDGE), High Speed Downlink Packet Access (HSDPA), Wideband Code Division Multiple Access (W-CDMA), Code Division Multiple Access (CDMA), Time Division Multiple Access (TDMA), Bluetooth®, Zigbee®, Wi-Fi, Voice over Internet Protocol (VoIP), LTE Advanced, IEEE802.16m, WirelessMAN-Advanced, Evolved High-Speed Packet Access (HSPA+), 3GPP Long Term Evolution (LTE), Mobile WiMAX (IEEE 802.16e), Ultra Mobile Broadband (UMB) (formerly Evolution-Data Optimized (EV-DO) Rev. C), Fast Low-latency Access with Seamless Handoff Orthogonal Frequency Division Multiplexing (Flash-OFDM), High Capacity Spatial Division Multiple Access (iBurst®) and Mobile Broadband Wireless Access (MBWA) (IEEE 802.20) systems, High Performance Radio Metropolitan Area Network (HIPERMAN), Beam-Division Multiple Access (BDMA), World Interoperability for Microwave Access (Wi-MAX), infrared communications and ultrasonic communication, etc., but is not limited thereto.

In one or more embodiments, the processor/processing unit 510 is communicatively coupled and communicates with a memory 530 where data and parameters are kept ready for use by the processing unit 510. The one or more memories 530 may comprise a selection of a hard RAM, disk drive, a floppy disk drive, a magnetic tape drive, an optical disk drive, a CD or DVD drive (R or RW), or other removable or fixed media drive.

First Control Unit Control Interface Windows

Before presenting data to a user the first control unit will receive, aggregate and process data from one or multiple sources such as the servo motor unit, the third control unit and the fourth control unit. The aggregated and processed data will further be displayed in non-overlapping, also referred to as disjunctive, control interface windows of the display or touchscreen area. These non-overlapping or disjunctive control interface windows of the display or touchscreen area will from hereon be referred to as control interface windows.

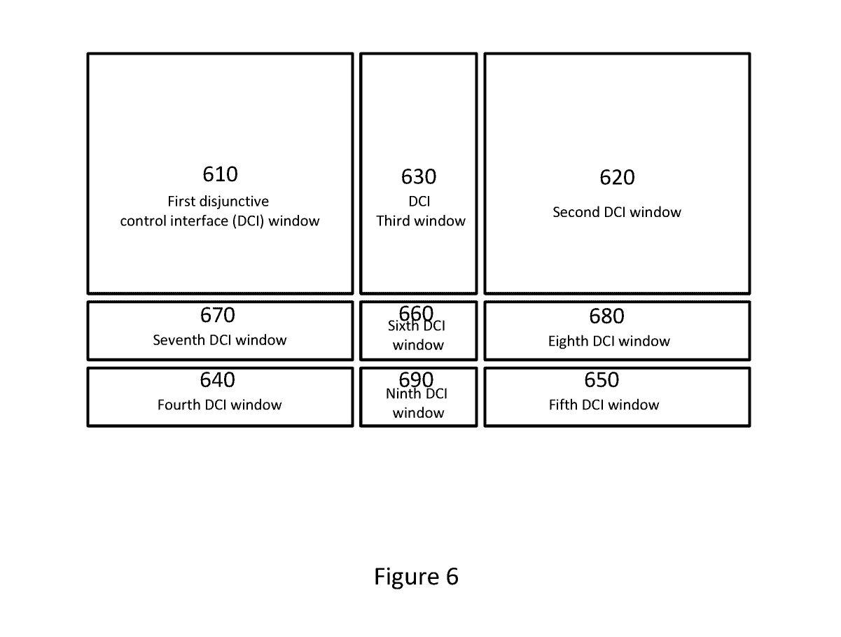

FIG. 6 shows a schematic view of a control interface comprising multiple disjunctive control interface windows 610-690. A first disjunctive control interface window 610 adapted to display a first set of display data obtained as function status data in the form of image data captured by said first X-ray system. A second control interface window 620 adapted to display a second set display data obtained as function status data in the form of image data captured by said second X-ray system. A third control interface window 6adapted to display a third set of display data obtained as functional data received from said memory in the form of a scrollable thumbnail list, wherein the functional status data comprises a thumbnail representation of captured X-ray image data and associated information. A fourth control interface window 64o adapted to display a fourth set of display data obtained as a first subset of said functional data received from said first X-ray system. A fifth control interface window 550 adapted to display a fifth set of display data obtained as a second subset said functional data received from said second X-ray system. a sixth control interface window 660 adapted to display a sixth set of display data obtained as a sixth subset of functional data received from said first X-ray system and from said second X-ray system and not part of said first or second subset. A seventh control interface window 670 adapted to display a seventh set display data obtained as function status data in the form of register data associated with said first X-ray system obtained from memory. An eighth control interface window 680 adapted to display a eighth set display data obtained as function status data in the form of register data associated with said second X-ray system obtained from memory. A ninth control interface window 690 adapted to display a ninth set of display data obtained as functional data received from a fourth control unit indicative of the position of transmitter/receiver pairs on the mobile unit and/or the relative position of the object being subjected or the X-ray-beam;

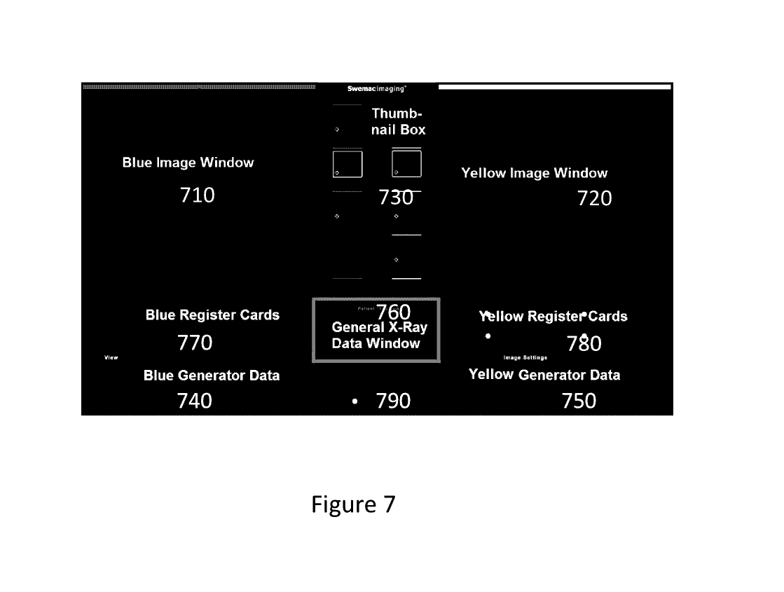

FIG. 7 shows an exemplary embodiment of a control interface comprising multiple disjunctive control interface windows as a first “Blue” image window 710, a second 720 “Yellow” image window, a thumb nail box 730, a “Blue” generator data window 740, a “Yellow” generator data window 750, a general X-ray data window 760, a “Blue” register card window 770, a “Yellow” register card window 780 and a positioning window 790.

In one or more embodiments of the invention, a mobile digital fluoroscopy system, having a mobile unit (1) having a first and a second X-ray system each having a transmitter and a receiver, said respective first and second X-ray systems being mounted on a G-arm to enable X-ray imaging in mutually intersecting planes;

comprising:

a first control unit configured with a control interface, wherein said control interface comprises multiple disjunctive control interface windows of the control interface adapted to display data based on display data.

In one or more embodiments, wherein said first control unit is further configured to:

receive functional status data as status control signals from a selection of a servo motor unit, a third control unit and a fourth control unit;

process function status data to a visual representation of said function status data as display data

present said visual representation based on said display data.

X-ray Images from Different X-ray Systems Simultaneously Displayed

When operating an X-ray imaging system with multiple X-ray systems it is essential not only to convey the most essential image information from each X-ray system but also to ensure that the combined information of two simultaneous X-ray sources are conveyed to a user controlling the X-ray imaging system via said control interface of said first control unit.

In one or more embodiments of the invention, a mobile digital fluoroscopy system, having a mobile unit (1) having a first and a second X-ray system each having a transmitter and a receiver, said respective first and second X-ray systems being mounted on a G-arm to enable X-ray imaging in mutually intersecting planes;

comprising:

a first control unit configured with a control interface, wherein said control interface comprises a first and a second disjunctive control interface window of the control interface adapted to display data based on display data.

wherein said first control unit is further configured to:

receive functional status data as status control signals from a first X-ray system and a second X-ray-system, wherein the functional status data comprises captured X-ray image data;

process function status data to a visual representation of said function status data as display data

present said visual representation based on said display data.

In one or more embodiments, wherein said display data is adapted to display said image data from said first X-ray system in said first disjunctive control interface window based on register data associated with said first X-ray system and display said image data from said second X-ray system is displayed in said second disjunctive control interface window based on register data associated with said first X-ray system.

X-ray Generator Control Interface Windows

When operating an X-ray imaging system with multiple X-ray systems it is essential to monitor and control the X-ray energy emitted, the X-ray radiation dose administered to an object, such as a patient as well as monitoring the load and temperature of the X-ray generator for each system. A transmitter configured with an X-ray generator in an X-ray system is controlled by transmitter generator control parameters such as X-ray examination type, X-ray dose, Snap shot mode on/off, number of X-ray image frames per second, X-ray generator pulse width, generator operating voltage (KV), generator operating current (mA), zoom mode, focus mode, automatic or manual operating current setting. Some transmitter generator control parameters are set individually for each generator and some control parameters are set common to both generators. A fourth set of transmitter generator control parameters unique to said first X-ray system is determined. A fifth set of transmitter generator control parameters unique to said second X-ray system is determined. A sixth set of transmitter generator control parameters not included in said fourth set and fifth set and common to both said first X-ray system and said second X-ray system is determined.

In one or more embodiments of the invention, a mobile digital fluoroscopy system, having a mobile unit (1) having a first and a second X-ray system each having a transmitter and a receiver, said respective first and second X-ray systems being mounted on a G-arm to enable X-ray imaging in mutually intersecting planes;

comprising:

a first control unit 510 configured with a control interface 520, wherein said control interface comprises a fourth 640, a fifth 650 and a Sixth 660 disjunctive control interface window of the control interface adapted to display data based on display data.

wherein said first control unit is further configured to:

receive functional status data as status control signals from a first X-ray system and a second X-ray-system, wherein the functional status data comprises generator control parameters;

process function status data to a visual representation of said function status data as display data

present said visual representation based on said display data.

In one or more embodiments, wherein said display data is adapted to

display a fourth subset of said functional data received from said first X-ray system in said fourth disjunctive control interface window,

adapted to display a fifth subset said functional data received from said second X-ray system in said fifth disjunctive control interface window

adapted to display a sixth subset of said functional data, comprising functional data received from said first X-ray system and from said second X-ray system and not part of said first or second subset, in said sixth disjunctive control interface window.

Image Sequence or Cine Loop

When operating an X-ray imaging system with multiple X-ray systems it is essential to monitor and control captured X-ray images or sequences/cine loops of captured X-ray images. The X-ray imaging system may capture individual X-ray images or multiple consecutive X-ray image sequences. The captured X-ray images or X-ray image sequences are saved to memory immediately after capture and associated with functional status data, wherein the functional status data comprises a time stamp, associated X-ray system, associated patient information and status information if the image is displayed in an image view control interface window in the control interface. The captured and saved are displayed by the first control unit in the control interface as a scrollable thumbnail list ordered by the associated timestamp. In one example, a first X-ray image captured by a first X-ray system simultaneously or nearly simultaneously as a second X-ray image captured by a second X-ray system is presented as a thumbnail pair in the scrollable thumbnail list, whereby images associated with the first X-ray system is displayed next to the control interface window displaying functional status data originating from said first X-ray system and images associated with the second X-ray system is displayed next to the control interface window displaying functional status data originating from said second X-ray system.

By default the latest captured X-ray image is displayed in the control interface window displaying functional status data originating from corresponding X-ray system. The user may also select a thumbnail in the thumbnail list and the first control unit will then process the user indication to present the X-ray image associated with the thumbnail.

The thumbnail associated with the X-ray image displayed in the control interface window displaying functional status data originating from corresponding X-ray system is adapted to indicate the association, e.g. by displaying a white frame.

The captured X-ray images or X-ray image sequences saved to memory may also be saved to a separate data base for patient files. For X-ray images or X-ray image sequences saved to memory but not yet transferred to the database, the associated thumbnail will be adapted to indicate no associated patient information, e.g. by displaying a red frame.

Examples of operations that may be performed on thumbnails in the thumbnail list are:

Expand thumbnail to any viewing area

Drag (Press, wait, move) thumbnail to image viewing area

Expand thumbnail to default viewing area

Touch (short press and lift) thumbnail

Scroll list of thumbnails up/down

Swipe (press, move, lift) up/down

a first control unit 510 configured with a control interface 520, wherein said control interface comprises a first, a second and a third disjunctive control interface window of the control interface adapted to display data based on display data.

In one or more embodiments, a mobile digital fluoroscopy system, having a mobile unit having a first and a second X-ray system each having a transmitter and a receiver, said respective first and second X-ray systems being mounted on a G-arm to enable X-ray imaging in mutually intersecting planes;

comprising:

a first control unit configured with a control interface and a memory, wherein said control interface comprises a first, a second disjunctive control interface window and a third disjunctive control interface window of the control interface adapted to display data based on display data.

wherein said first control unit is further configured to:

receive a user indication as user input data indicative of an operation on a scrollable thumbnail list displayed in said first control interface window, wherein thumbnails in said thumbnail list is associated by functional status data to X-ray images or X-ray image sequences captured by said first and second X-ray system and saved to said memory;

In one or more embodiments, a mobile digital fluoroscopy system, having a mobile unit having a first and a second X-ray system each having a transmitter and a receiver, said respective first and second X-ray systems being mounted on a G-arm to enable X-ray imaging in mutually intersecting planes;

comprising:

a first control unit configured with a control interface and a memory, wherein said control interface comprises a first, a second disjunctive control interface window and a third disjunctive control interface window of the control interface adapted to display data based on display data.

wherein said first control unit is further configured to:

receive a user indication as user input data indicative of an operation on a scrollable thumbnail list displayed in said first control interface window, wherein thumbnails in said thumbnail list is associated by functional status data to X-ray images or X-ray image sequences captured by said first and second X-ray system and saved to said memory;

In one or more embodiments, wherein said first control unit is further configured to:

retrieve functional status data from memory, wherein the functional status data comprises a thumbnail representation of captured X-ray image data and associated information;

process function status data to a visual representation of said function status data as display data, wherein said visual representation comprises a scrollable thumbnail list.

present said visual representation based on said display data.

In one or more embodiments, wherein said operation is a selection of:

clicking said thumbnail thereby triggering the first control unit presenting an associated X-ray image in said third control interface window;

Dragging said thumbnail in said first control interface window to said second disjunctive control interface window, thereby triggering the first control unit presenting an X-ray image associated to said thumbnail in said second control interface window

swiping said thumbnail up or down, thereby triggering said first controller to scroll the thumbnail list; In one or more embodiments, wherein the functional status data comprises a selection of a time stamp, an associated X-ray system, associated patient information and status information if the image is displayed in an image view control interface window in the control interface.

### USE CASE EMBODIMENTS

Register data display

This register data includes basic image “processing” functions,

#16 Image flip Vertical toggle button

This button will flip the respective image including the cine vertically. A second tap on the button reverses the image orientation again (thus back to start).

#17 Image flip horizontal toggle button

This button will flip the respective image including the cine horizontally. A second tap on the button reverses the image orientation again (thus back to start).

#18 Rotate image CW button Pan Image

When pressed the image will rotate CW or CCW with a high resolution of about 1° per movement by rotating two fingers on the respective screen. Holding the button down will result in a continuous CW rotation.

#19 Rotate image CCW button

When pressed the image will rotate CW or CCW with a high resolution of about 1° per movement by rotating two fingers on the respective screen. Holding the button down will result in a continuous CCW rotation.

#20 Auto collimator (future function) toggle button/Reset collimator button

When button is active a function will move the collimators to filter out as much direct radiation as possible, i.e. smallest possible setting. If pressed, the collimators will move to their reset position (outside image area (no collimation)). The user can still collimate manually.

#21 Zoom in button

When pressed the image will zoom up (enlarge) by a determined factor. (+25%). When pressed several times the image will continue to zoom up in steps.

The 2 finger smooth zoom method is always accessible. A one finger double tap will increase or decrease by a factor of 2 (toggle function)

Holding 1 finger on the screen will allow to pan the image with the finger movement. Also always accessible

#22 Zoom out button

When pressed the image will zoom down (decrease size) by a determined factor. (2×)

The 2 finger smooth zoom method is accessible after tapping on the icon below. A one finger double tap will increase or decrease by a factor of 2 (toggle function).

Holding 1 finger on the screen will allow to pan the image with the finger movement.

#23 Invert (negative) image toggle button

This button toggles between B/W and W/B

#24 Print image button

When pressed the image will be printed on the internal printer.

#25 Save image button

When pressed the onscreen image will be saved to the storage disc (Patient file).The image will be stored in the patient study under the respective time (as a name).

In case of dual images both images are stored by hitting one of the button on either program side after a prompt: “Should both images be stored? yes or no”. Please store accordingly. If “no” only the image on the side that was chosen must be stored.

#26 Save Cine button

When pressed the current cine/sequence will be saved to the storage disc (Patient file). The cine series will be stored in the patient study under the respective time (as a name) with a c in front of the name to designate a cine series.

In case of a dual cine both cines are stored by hitting one of the button on either program side after a prompt: “Should both cines be stored? yes or no”. Please store accordingly. If “no” only the cine on the side that was chosen must be stored.

Register data Image Settings:

All functions are ghosts as long as no images have been collected.

After pressing image settings the image processing tab the controls are called up on the register card on the touch screen. The box includes all the image processing controls as displayed:

#27 Enhance Contrast toggle button

When this button is toggled on (enhanced) a contrast filter will be applied to the image.

The image edge enhance contrast function can be applied to any image in the main image window. If a cineloop is used the enhancement applies to all images. If the system is in live mode the enhancement applies to all images.

To use the function click on the respective icon. Clicking on another image will change the selection. The result is directly visible after clicking the respective icon. A second click on this icon returns the image to its original state.

This function increases contrast and accentuate detail in the image, but may also accentuate noise. This filter uses the weighting factors ( )to replace each pixel with a weighted average of the 3×3 neighborhood. The function is a toggle function when enhancement is on or off.

The resulting image can be viewed and printed or stored

#28 Edge Enhance toggle button

When this button is toggled on an edge enhancement filter will be applied.

The image edge enhancement function can be applied to any image in the main image window. If a cineloop is used the enhancement applies to all images. If the system is in live mode the enhancement applies to all images.

To use the function click on the respective icon. Clicking on another image will change the selection. The result is directly visible after clicking the respective icon. A second click on this icon returns the image to its original state.

This function uses a Sobel edge detector to highlight sharp changes in intensity.

Two 3×3 convolution kernels (and ( )are used to generate vertical and horizontal derivatives. The final image is produced by combining the two derivatives using the square root of the sum of the squares.

The resulting image can be viewed and printed or stored.

Noise reduction slider (smooth)

When this button is moved a noise reduction filter will be applied to the image. The further it is moved right the higher the degree of noise reduction. The noise reduction filter (frame averaging) on the MX300 board will be used.

The smooth function can be applied to any image in the main image window. If a cineloop is used the enhancement applies to all images. If the system is in live mode the enhancement applies to all images.

To use the function click on the respective icon. Clicking on another image will change the selection. The result is directly visible after clicking the respective icon. A second click on this icon returns the image to its original state.

This function smoothens detail in the image selected. This filter uses the weighting factors ( ) to replace each pixel with the average of its 3×3 neighborhood. The further the slider is moved to the right the higher the smoothing. The weight factors used must be tested.

The resulting image can be viewed and printed or stored.

Brightness slider

Slider will change the brightness setting.

Contrast slider

Slider will change the contrast setting.

White Level Slider

Slider will change the white level setting.

This function can enhances the contrast in darker areas while reducing the contrast in lighter areas depending on the LUT settings. This function is used to adapt the image contrast to the user's perception.

The contrast change is achieved by applying a non-linear transfer curve (e.g. represented by a LUT). The grey values of the image are remapped using the non-linear transfer curve. The degree of white compression, i.e. non-linearity, is steered by user. The result of the contrast change is directly visible in the image.

The image including the changes can be viewed and printed and stored.

Register data Annotation function

#29 Line/Ruler Tool Button

When pressed a line can be drawn with the following functions:

Adjustments to the line can be made by dragging the either end of the line

If the image was calibrated (see #31), the actual length (in cm) of the newly drawn line is written over the line.

If a second line is drawn, the angle between these two lines is also displayed near the vortex of the lines, together with the corresponding lengths for both lines. If the lines do cross (no automatic extension calculations) the angle is not displayed.

Thus, when the icon is tapped a line will appear within the image. Adjustments to the line can be made by moving the line ends (change in orientation and size) or by touching the center of the and thus moving the line on the image surface.

#30 Text button

When pressed the Note tool will be shown. Position of the note can be adjusted be dragging the note.

Thus, cursor will start to blink inside the image and then the user can add text to the image.

The onscreen keyboard appears. As second tap to the icon will end data input (The onscreen keyboard disappears) but will then allow to move the text over the screen. If no movement occurs for 5 seconds the movement function ends.

#31 Add reference (Image calibration)

When pressed the image can be calibrated by the following procedure:

1. Draw a line of known length into the image.

2. Enter the known length in the “Length” field box that should then pop up along with the onscreen keyboard or a number pad.

a. As the cine images are all of one size any of the images in a loop can be used for calibration. The calibration is valid for all images in that loop

b. Re-calibration is possible.

c. Existing measurements are updated if a re-calibration action is performed.

d. For next loop (or single image) the calibration shall still remain until the user decides to recalibrate

#32 Remove Marker

When pressed an object can be selected and removed (deleted) by tapping the object. After one object is deleted the function will be disabled. To remove a second item it must be activated again

Register data Image sequence or Cine Replay

All functions are ghosts as long as no images have been collected.

After pressing cine replay the cine register card is opened on the touch that includes all the cine functions. In general, the cineloop will be collecting images in the back ground as long as the system is in live mode. The number of images in the loop depends upon the size or the RAM memory.

If the cine replay tab is clicked live image display will be terminated and the cine function will be opened.

As soon as the maximum number of images have been collected the oldest images will be overwritten automatically without a query. When live image acquisition is stopped no more images will be loaded into the cineloop and the last acquired will be displayed (last image hold). This image will have the lowest image number in the loop, i.e. 1.

When the cine window is closed the cine display will continue in its last state. In some cases the last image displayed may be held (in case of measurements for example). If the loop is stopped in some way it will not go back to the last state after the respective function was terminated but will display the respective image on screen at the time.

The cine functions are:

Sequence position slider (Image Number)

The touch button slider is used to navigate through the sequence of images.

#33 Frame skip button (Forward)

When the cine is stopped/paused: When pressed the image displayed will go forward one frame

When pressed for an extended period the image displayed will go forward frame by frame

#34 Play button

When pressed the cine loop will start to play. If pressed again re-play will be paused/stopped.

The speed should be the same as the recording speed.

#35 Frame skip button (Back)

The cine is stopped/paused: When pressed the image displayed will go back one frame

Eight control interface window or thumbnail area

The thumbnail area lies between the two image windows and contains thumbnails that belong to cineloops or individual images that were transferred intermediately (or were recalled from the patient file) for later evaluation. A cine series carries an image number and a time stamp (the time when live imaging was stopped). A single image does not have an image number but only a time stamp. The thumbnail images carry a side designator (blue or yellow bar above the thumbnail) that defines to which imaging chain the image(s) belongs. The side designator must be stored for later retrieval because when the images are loaded to the thumbnail area from memory they must be placed onto the correct thumbnail side and carry the correct designator (yellow or blue).

Thumbnails or images that belong to the same time stamp must be displayed next to one another joined by a. As live image start/stop is footswitch controlled it is known which images belong to the same time stamp. 3 footswitches exist: One for the yellow and one for the blue imaging line and one for both. Thus if the “both” peddle is used to stop live imaging the images (yellow and blue) obviously belong together and can be stored together. If they are stored separately we will not take it for granted that they belong together. They are then treated as separate cines or separate images. Still, the yellow and blue designator must be saved.

There are also two buttons on the footswitch unit. These buttons have the same function as the “Save Image” in the “View” register card, one for blue and one for yellow plane.

Cines always carry a Icon 74 so they can be easily recognized.

Thus, when loading images or cines from the patient file to the thumbnail area the will be placed if they have the same time stamp. Images and cines with different time stamps will be treated as separate images.

Obviously, it can occur that more thumbnail have been created than fit into the thumbnail area. In such a case the usual Windows slide bar will appear and the thumbnails can be scrolled.

The thumbnail in the respective thumbnail box belongs to the image(s) displayed in the respective control interface window as far as it was manually selected for display and loaded from the box. That means that images that are currently being acquired (i.e., live images) and displayed in the main windows are not displayed as thumbnails until image acquisition has been stopped. As soon as live imaging has been stopped the image or a cine series will be transferred to the thumbnail. The images are now available for later revue. Those that have not yet been transferred to the patient file are designated by a red box. The images that have been saved to or been loaded from the patient file do not have a red box around them. The image or cines displayed in the main windows are designated by a white box (as long as they are not in live mode). A combination of a white box and a red box is possible.

Icon 70 indicates that the image/cine has been saved to the patient file

An image number (the number of images in the cine) is displayed below the respective thumbnail together with the time (HH:MM:SS) when the cineloop was acquired (last image taken).

No image may be deleted from a thumbnail series. Only the series as a whole may be deleted.

The patient name is displayed below the thumbnail box.

Image transferred to the thumbnail area but not saved to the patient file prior:

A cine or image that was stored prior cannot be overwritten in the patient file. In case the user intends to store the images or cines the images must first be transferred to the image window and stored via the from the register card View.

In all cases the user must store the images/cines separately in the patient file. They are listed by their time stamp and “color” blue or yellow.

The images in a cine series or a single image thumbnail can be transferred to the main image windows by just tapping on the respective thumbnail. Any images or image series in the main windows will then be removed but not deleted from the thumbnails and the respective image or series is loaded.

Even in case of a dual cine or dual images both cines or images must be transferred to the main images separately.

It is also possible to load a blue image or series on the yellow image side and vice versa. This function can be used to compare a blue with another blue image or cine series for example. In order to facilitate this the user will sweep the respective thumbnail in the direction of the blue or yellow image frame. This will not change the image registration as a blue or yellow image.

If a blue image is displayed in the yellow window the yellow header bar should change its color. The same is true for the blue frame respectively.

If the image or series has not been stored prior and is done now it will be stored under its original color.

If an image series has already been stored and the user wishes to store it again he must b prompted:

“This image/image series has already been stored. If you wish to store it again please enter the file name here and then tap save or else tap no:” After entering the file the user must tap the save tab. The on-screen keyboard must pop up of course as well.

Exiting a Study and Returning to the Patient Management System

When a study is ended the system may be in a number of different states:

1. Images and cines may have been transferred to the thumbnail area but not saved.

2. Pre-saved images or cines may have been loaded to the thumbnail area and transferred to the main windows.

3. Pre-saved images or cines may have been loaded to the thumbnail area and not transferred to the main windows.

4. Other combinations are possible.

It is the objective not to loose any image information except if the user confirms that he does not wish to save specific images or image series in the patient file. The area beneath the thumbnail area can be used to terminate a study:

That area includes the patient name. By tapping the End session Icon 76, the system change screen to the “Evaluate screen”. This is a screen where all images/cines from the last session are displayed, using the entire screen area for further evaluation. Here the user has the following options:

Resume session Return to main screen and continue last session

Evaluate images from last session, both saved and not saved will be visible.

Images may be viewed in full/half/quarter/eight screen for evaluation

Images and cines can be saved to patient file in DICOM. Icon 70 will indicate a saved image/cine

Images and cines can be saved internally in the PC without DICOM connection

Images and Cines can be printed on the internal printer

Images and cines can be exported to a USB memory card

Create dose report and view on display or internal printer

Return to patient management system “New session”

There is also a possibility to delete images from the patient file. This function is mainly used to clean up the hard drive if it is getting full.

Shut down the system

See flowchart “GUI Flowchart PM oriented 20131105.PDF”

It would be nice if one short press on the power button during surgery would do the same thing as “End session”

1.3. Loading or Images or Cine Series to the Thumbnail Area and Deleting Images from an Exam

As discussed, this function is not really needed during surgery.

The area beneath the thumbnail area can be used to load images from a study to the thumbnail area:

That area includes the patient name.

The user accesses the study to load images by tapping. In this case the images inside the respective study will be displayed. The exams are listed by date and time when they were generated and listed in the yellow or blue imaging chain.

In all cases the image series in the same line will be loaded (if 2 are present). Image series can be deleted by clicking delete after selecting the series by clicking the respective box in front of the image series.

Individual generator settings

First and second generator settings are accessible/displayed here. This is an area specific for the respective generator settings and generator information.

In this field the session time is shown as well as total dose received to the patient.

It is also possible to select Snapshot (ER) mode and Frame rate.

The generator data is displayed at the top of the 2 viewing monitors, i.e. above the image field and on the touch screen below the register cards on the respective side.

The following functions are available:

#37 kV increase button

When pressed the button will increase the kV in steps of 1 kV per tap. If the button is pushed and held for more than two seconds the generator will start increasing the kVs in steps of 1 kV every 0.2 s (configurable in the service menu).

#38 kV Setting Value

The actual kV values are displayed (Range: 40-110 kV) in both manual and automatic mode. This function is always active

#39 kV Decrease button

When pressed the button will decrease the kV in steps of 1 kV per tap. If the button is pushed and held for more than two seconds the generator will start decreasing the kVs in steps of 1 kV every 0.2 s (configurable in the service menu).

#40 Auto/Manual Generator Setting Toggle Button.

If button is in auto mode displaying the auto icon the generator will control the kV and mA settings based on the APR used.

If button is in manual mode displaying the “hand” the generator will use the kV set by the user and shown in the display.

#41 Current Dose Rate Info Text Field

The dose rate is the calculated dose using the current settings displayed.

This function is always active (Arial Bold, n pixels).

#42 Accumulated Dose Info Text Field

The accumulated dose is the calculated or measured dose is displayed.

This function is always active (Arial Bold, n pixels).

#43 Tube Heat Indicator

This indicator shows the accumulated heat of the tube. (good to know if you plan to use fluoroscopy for a long time and if the tube needs to cool down). The scale will display three stages (cool-medium-hot)

Control parameters common to the first and second transmitter

The common generator settings/information and timer functions are displayed beneath the thumbnail area. APRs (Application Specific Setups) include all the generator settings and other issues:

APRs Used:

procedure code Examination type LDF/HDF Dose Snap shot

| | | |
|---|---|---|
| 5 × 20 msec Frames per second | Pulse width (msec) | KV start mA |
| Zoom1/2/3 Focus S/L | Auto/manual | Varian Detector Mode |

#44 Reset 5 min Alarm Button

This button resets the alarm that is triggered the patient has been illuminated for a period of 5:00 min in total. Thus the internal counter is stopped when n X-rays are emitted, i.e. the generator power is zero. When the time of 5 min has passed an alarm will sound and the button will blink at 1 Hz. When the button is tapped the clock will be reset to 0 and the function will begin again from start

#48 Time Indicator Display

This field shows total X-ray time (when generators output X-ray) (illumination of the patient) displayed in Minutes : Seconds

#45 End session/Change application button

This function changes the application (APR). The icon displays the selected APR

#46 Dose Level Setting Buttons

The button toggles between normal and high dose settings. The high level is a predefined value.

#47 Snapshot toggle button

This button sets the system into Snapshot mode when activated. That means only one image will be taken when stepping on the footswitch(s)

#47 Pulsed fluoroscopy pulse rate.

This slider indicates and allows the user to change the pulse rate (frames/second). When changing APR the slider is set to whatever is decided in the APR. The user is able to override the APR setting by using the slider

There is a typo in the image. The selectable framerates are: 3 6 12 30

Fourth control functional data

This button opens a window where the pillars of the stand can be raised or lowered and where the II lift can be moved. The motor screen is position beneath the thumbnail area: Help Function

The help function is located next to the motor screen. When this icon is tapped a manual will be opened. The manual will be a PDF help file.

In one or more embodiments, a non-transitory computer readable memory on which is stored computer readable code configured to, when executed in a processor, perform any or all of the method steps described herein.

A tangibly embodied computer-readable medium including executable code that, when executed, causes a first control unit to perform any or all of the method steps described herein.

A tangibly embodied computer-readable medium including executable code that, when executed, causes a servo motor unit to perform any or all of the method steps described wherein.

1. A mobile digital fluoroscopy system, having a mobile unit (1) having a first and a second X-ray system each having a transmitter (21, 23) and a receiver (22, 24), said respective first and second X-ray systems being configured to enable X-ray imaging in mutually intersecting planes, comprising:
a first control unit (510) configured with a control interface (520) and a memory (530), wherein said control interface comprises a plurality of disjunctive control interface windows of the control interface adapted to display controls configured to control functions of the fluoroscopy system and to display data based on functional status data of said first and second X-ray systems.

2. The system of claim 1, wherein said first control unit is further configured to:
receive functional status data as status control signals from receivers (22, 24),
process function status data to a visual representation of said function status data as display data
present said visual representation on said control interface 520 based on said display data, wherein presenting comprises displaying subsets of display data in said multiple disjunctive control interface windows (610-690).

3. The system of any of the preceding claims, wherein said multiple disjunctive control interface windows comprises:
a first disjunctive control interface window adapted to display a first set of display data obtained as function status data in the form of image data captured by said first X-ray system,
a second control interface window adapted to display a second set display data obtained as function status data in the form of image data captured by said second X-ray system, and
a third control interface window adapted to display a third set of display data obtained as functional data received from said memory in the form of a scrollable thumbnail list, wherein the functional status data comprises a thumbnail representation of captured X-ray image data and associated information.

4. The system of any of the preceding claims, wherein said multiple disjunctive control interface windows comprises:
a fourth control interface window adapted to display a fourth set of display data obtained as a first subset of said functional data received from said first X-ray system.

5. The system of any of the preceding claims, wherein said multiple disjunctive control interface windows comprises: a fifth control interface window adapted to display a fifth set of display data obtained as a second subset said functional data received from said second X-ray system.

6. The system of any of the preceding claims, wherein the system further comprises a third control unit (512) configured to determine the X-ray energy emitted by the transmitters based on predefined data parameters retrieved from a memory 532 communicatively coupled to said third control unit and functional status data as user input data received from said first control unit 510, to send functional status data indicative of X-ray beam transmission to said first control unit (510) and to determine functional status data indicative of the X-ray beam dose administered to an object, .e.g. a patient, wherein said multiple disjunctive control interface windows comprises: a sixth control interface window adapted to display a sixth set of display data obtained as a sixth subset of functional data received from said first X-ray system and from said second X-ray system and not part of said first or second subset.

7. The system of any of the preceding claims, wherein said control interface further comprises a seventh control interface window adapted to display a seventh set display data obtained as function status data in the form of register data associated with said first X-ray system obtained from memory.

8. The system of any of the preceding claims, wherein said control interface further comprises an eighth control interface window adapted to display a eighth set display data obtained as function status data in the form of register data associated with said second X-ray system obtained from memory

9. The system of any of the preceding claims, wherein the system further comprises a fourth control unit (511) configured to receive functional status data as control signals from said first control unit, wherein the functional status data is based on processed user input data, to control servo motors to a predetermined position based on said functional status data by sending servo motor signals, thereby positioning the transmitter/receiver pairs on the mobile unit or the object being subjected or the X-ray-beam.

10. The system of any of the preceding claims, further comprises servo motor functional status data indicative of the status, e.g. position of a servo motor, of a servo motor is obtained by the fourth control unit 511 by receiving servo motor signals and to send functional status data as status control signals to said first control unit, wherein said multiple disjunctive control interface windows comprises: wherein a ninth control interface window adapted to display a ninth set of display data obtained as functional data received from a fourth control unit indicative of the position of transmitter/receiver pairs on the mobile unit and/or the relative position of the object being subjected or the X-ray-beam;

11. The system of any of the preceding claims, further comprising:
a dose measuring device mounted on each respective X-ray transmitter and configured to measure emitted dose of X-ray radiation from said respective X-ray transmitter, wherein said third control unit (512) is further configured to calculate a dose of X-ray radiation received by an object from said respective X-ray transmitter and according to one or more selected dose indication methods.

12. A method in a mobile digital fluoroscopy system, having a mobile unit (1) having a first and a second X-ray system each having a transmitter (21, 23) and a receiver (22, 24), said respective first and second X-ray systems being mounted on a G-arm (18) to enable X-ray imaging in mutually intersecting planes, comprising the steps of:
receiving functional status data as status control signals from a selection of said memory, a servo motor unit, a third control unit and a fourth control unit;
processing function status data to a visual representation of said function status data as display data;
presenting said visual representation based on said display data.

13. A computer program product comprising computer readable code configured to, when executed in a processor, perform any or all of the functions or method steps of any of the preceding claims.

14. A non-transitory computer readable memory on which is stored computer readable code configured to, when executed in a processor, perform any of the preceding claims.

15. A computer program product comprising computer readable code configured to, when executed in a processor, perform any or all of the functions or method steps described herein.

16. A non-transitory computer readable memory on which is stored computer readable code configured to, when executed in a processor, perform any or all of the functions or method steps described herein.
